(19) United States
(12) Patent Application Publication
(10) Pub. No.: US 2018/0249921 A1
(43) Pub. Date: Sep. 6, 2018

(54) ELECTROCARDIOGRAM ANALYZING METHOD, ELECTROCARDIOGRAM ANALYZING APPARATUS, ELECTROCARDIOGRAM ANALYZING PROGRAM, AND COMPUTER-READABLE MEDIUM STORED WITH THE ELECTROCARDIOGRAM ANALYZING PROGRAM

(71) Applicant: NIHON KOHDEN CORPORATION, Shinjuku, Tokyo (JP)

(72) Inventors: Yoshinobu ONO, Shinjuku-ku, Tokyo (JP); Tsuneo TAKAYANAGI, Shinjuku-ku, Tokyo (JP); Takashi KAIAMI, Shinjuku-ku, Tokyo (JP)

(21) Appl. No.: 15/758,221

(22) PCT Filed: Aug. 29, 2016
(86) PCT No.: PCT/JP2016/003928

§ 371 Date: Mar. 7, 2018

(30) Foreign Application Priority Data
Sep. 10, 2015 (JP) 2015-178764

(51) Int. Cl. A61B 5/0468; A61B 5/04; A61B 5/0456; A61B 5/02; A61B 5/00

(52) U.S. Cl. CPC A61B 5/0468; A61B 5/04014; A61B 5/4035; A61B 5/02028; A61B 5/0456

(57) ABSTRACT

An electrocardiogram analyzing method includes: (a) acquiring electrocardiogram data representing an electrocardiogram waveform having heart beat waveforms; (b) acquiring RR interval data from the electrocardiogram data; (c) detecting at least one premature ventricular contraction causing a compensatory pause; (d) extracting, from the RR interval data, RR intervals during the detected premature ventricular contraction and before and after the detected premature ventricular contraction; (e) extracting a first RR interval group and a second interval group respectively from the RR intervals, wherein the first interval group includes the intervals before the premature ventricular contraction, and the second interval group includes the intervals after the premature ventricular contraction; (f) performing a predetermined frequency analysis on the first RR interval group; (g) performing the frequency analysis on the second RR interval group; and (h) comparing a first analysis result obtained by the step (f) and a second analysis result obtained by the step (g).

### TECHNICAL FIELD

The present disclosure relates to an electrocardiogram analyzing method. In addition, the present disclosure also relates to an electrocardiogram analyzing apparatus, an electrocardiogram analyzing program, and a computer-readable medium stored with the electrocardiogram analyzing program.

### BACKGROUND

U.S. Pat. No. 6,496,722 discloses a method for inspecting an autonomic nerve function such as baroreflex of a patient using HRT (Heart Rate Turbulence) obtained from an electrocardiogram waveform to thereby predict a sudden death after myocardial infarction or due to cardiac failure. Here, HRT means a variation of a sinus rhythm immediately after appearance of a premature ventricular contraction (hereinafter referred to as PVC) causing a compensatory pause.

U.S. Pat. No. 6,496,722 further discloses that a graph representing the relationship between heart beat numbers immediately after the PVC and RR intervals corresponding to time intervals between adjacent ones of the heart beats is acquired from the electrocardiogram waveform and the graph is analyzed on a time area by an HRT analysis method using TO (Turbulence Onset), TS (Turbulence Slope) etc. Here, TO expresses a shortened amount of each RR interval after the PVC, and TS expresses the speed for elongation of the RR interval.

However, in the HRT analysis method disclosed in U.S. Pat. No. 6,496,722, TO and TS are used as two evaluation parameters. Accordingly, when, for example, one of the evaluation parameters indicates a normal value while the other evaluation parameter indicates an abnormal value, there is a possibility that doubt may occur in the diagnosis result. In addition, the baroreflex etc. is evaluated based on analysis on the variation in the RR intervals before and after the PVC. The HRT analysis method using TO does not directly evaluate the variation in the RR intervals but simply evaluates a shortened amount of each RR interval.

### SUMMARY

An aspect of the present disclosure provides an electrocardiogram analyzing method by which an autonomic nerve function such as baroreflex of a patient can be inspected relatively easily. In addition, other aspect of the present disclosure provides an electrocardiogram analyzing apparatus for carrying out the electrocardiogram analyzing method, an electrocardiogram analyzing program, and a computer-readable medium stored with the electrocardiogram analyzing program.

According to a first aspect of the present disclosure, the electrocardiogram analyzing method comprises:

(a) acquiring electrocardiogram data representing an electrocardiogram waveform having heart beat waveforms occurring continuously on a time axis;
(b) acquiring RR interval data from the electrocardiogram data, wherein the RR interval data includes RR intervals, and each of the RR interval represents a time interval between adjacent ones of the heart beat waveforms;
(c) detecting at least one premature ventricular contraction causing a compensatory pause;
(d) extracting, from the RR interval data, RR intervals during occurrence of the detected premature ventricular contraction and before and after the occurrence of the detected premature ventricular contraction;
(e) extracting a first RR interval group and a second interval group respectively from the RR intervals, wherein the first RR interval group includes the RR intervals before the occurrence of the premature ventricular contraction, and the second RR interval group includes the RR intervals after the occurrence of the premature ventricular contraction;
(f) performing a predetermined frequency analysis on the first RR interval group;
(g) performing the predetermined frequency analysis on the second RR interval group; and
(h) making a comparison between a first analysis result obtained by the step (f) and a second analysis result obtained by the step (g).

According to a second aspect of the present disclosure, the electrocardiogram analyzing apparatus.

The apparatus comprises:

an electrocardiogram data acquiring section configured to acquire electrocardiogram data representing an electrocardiogram waveform having heart beat waveforms occurring continuously on a time axis;
an RR interval data acquiring section configured to acquire RR interval data from the electrocardiogram data, wherein the RR interval data includes RR intervals, and each of the RR interval represents a time interval between adjacent ones of the heart beat waveforms;
a detector configured to detect at least one premature ventricular contraction causing a compensatory pause;
a first extractor configured to extract, from the RR interval data, RR intervals during occurrence of the detected premature ventricular contraction and before and after the occurrence of the detected premature ventricular contraction;
a second extractor configured to extract a first RR interval group and a second interval group respectively from the RR intervals, wherein the first RR interval group includes the RR intervals before the occurrence of the premature ventricular contraction, and the second RR interval group includes the RR intervals after the occurrence of the premature ventricular contraction;
a first analyzer configured to perform a predetermined frequency analysis on the first RR interval group;
a second analyzer configured to perform the predetermined frequency analysis on the second RR interval group; and
a comparing section configured to make a comparison between a first analysis result obtained by the first analyzer and a second analysis result obtained by the second analyzer.

According to a third aspect of the present disclosure, the electrocardiogram analyzing program causes a computer to perform operations comprising:

(a) acquiring electrocardiogram data representing an electrocardiogram waveform having heart beat waveforms occurring continuously on a time axis;
(b) acquiring RR interval data from the electrocardiogram data, wherein the RR interval data includes RR intervals, and each of the RR interval represents a time interval between adjacent ones of the heart beat waveforms;
(c) detecting at least one premature ventricular contraction causing a compensatory pause;
(d) extracting, from the RR interval data, RR intervals during occurrence of the detected premature ventricular contraction and before and after the occurrence of the detected premature ventricular contraction;
(e) extracting a first RR interval group and a second interval group respectively from the RR intervals, wherein the first RR interval group includes the RR intervals before the occurrence of the premature ventricular contraction, and the second RR interval group includes the RR intervals after the occurrence of the premature ventricular contraction;
(f) performing a predetermined frequency analysis on the first RR interval group;
(g) performing the predetermined frequency analysis on the second RR interval group; and
(h) making a comparison between a first analysis result obtained by the step (f) and a second analysis result obtained by the step (g).

### BRIEF DESCRIPTION OF DRAWINGS

FIG. 1 is a hardware configuration diagram illustrating an electrocardiogram analyzing apparatus according to an embodiment of the present disclosure.

FIG. 2 is a diagram illustrating functional blocks of a controller.

FIG. 3 is a flow chart for illustrating an electrocardiogram analyzing method according to an embodiment of the present disclosure.

FIG. 4A illustrates the relationship between plural RR intervals in the vicinities of respective plural premature ventricular contractions and orders of heart beat waveforms (hereinafter referred to as beat numbers).

FIG. 4B illustrates the relationship between the RR intervals averaged for each of the beat numbers and the beat numbers.

FIG. 5 illustrates a first RR interval group and a second RR interval group.

FIG. 6 illustrates a power spectrum of the first RR interval group and a power spectrum of the second RR interval group.

FIG. 7A is a reference graph illustrating the relationship between plural RR intervals in the vicinities of respective premature ventricular contractions and beat numbers.

FIG. 7B is a reference graph illustrating the relationship between the RR intervals averaged for each of the beat numbers and the beat numbers.

FIG. 8 is a reference graph illustrating a first RR interval group and a second RR interval group.

FIG. 9 is a reference graph illustrating a power spectrum of the first RR interval group and a power spectrum of the second RR interval group.

### DESCRIPTION OF EMBODIMENTS

An embodiment of the present disclosure will be described below with reference to the drawings. Incidentally, description about elements having the same reference signs as those which have been already described will be omitted in description of the embodiment for convenience of explanation.

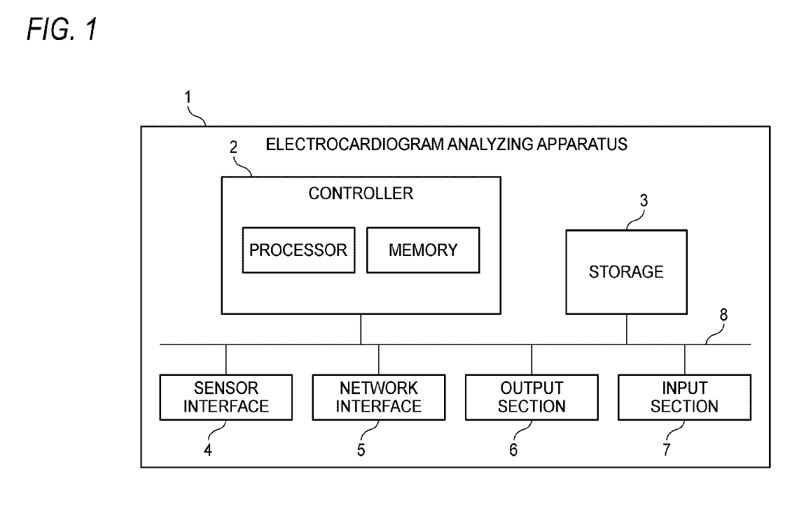

FIG. 1 illustrates a hardware configuration diagram of an electrocardiogram analyzing apparatus 1 according to an embodiment of the present disclosure. As shown in FIG. 1, the electrocardiogram analyzing apparatus 1 can include a controller 2, a storage 3, a sensor interface 4, a network interface 5, an output section 6, and an input section 7. These are connected to one another communicably through a bus 8.

Although the electrocardiogram analyzing apparatus 1 is a dedicated apparatus for analyzing an electrocardiogram waveform, it may be, for example, a personal computer, a smartphone, a tablet, or a wearable device such as an Apple Watch.

The controller 2 can include a memory and a processor. For example, the memory can include an ROM (Read Only Memory) in which various programs etc. have been stored, an RAM (Random Access Memory) having plural work areas where various programs etc. to be executed by the processor can be stored. For example, the processor is a CPU (Central Processing Unit) which is configured to load a designated program from the various programs incorporated in the ROM onto the RAM and execute various processes in cooperation with the RAM.

The controller 2 may control various operations of the electrocardiogram analyzing apparatus 1, particularly when the processor loads an electrocardiogram analyzing program which will be described later on the RAM and executes the electrocardiogram analyzing program in cooperation with the RAM. The controller 2 and the electrocardiogram analyzing program will be described below in detail.

For example, the storage 3 is a storage device such as an HDD (Hard Disk Drive), an SSD (Solid State Drive) or a flash memory, which is configured to store programs or various data. The electrocardiogram analyzing program may be incorporated in the storage 3. In addition, electrocardiogram data acquired by a not-shown electrocardiogram sensor may be stored in the storage 3. The electrocardiogram sensor is configured to acquire a weak electric signal generated from the heart of a patient in order to generate electrocardiogram data. Here, the electrocardiogram data represent an electrocardiogram waveform having heart beat waveforms (QRS waveforms etc.) occurring continuously on a time axis.

The sensor interface 4 can be configured to connect the electrocardiogram analyzing apparatus 1 to the electrocardiogram sensor communicably. For example, the electrocardiogram data acquired by the electrocardiogram sensor are transmitted to the controller 2 or the storage 3 through the sensor interface 4. The sensor interface 4 may have an A/D converting function.

The network interface 5 can be configured to connect the electrocardiogram analyzing apparatus 1 to a not-shown communication network. Here, the communication network can include an LAN (Local Area Network), a WAN (Wide Area Network) or the Internet etc. For example, an analysis result outputted from the controller 2 may be transmitted to another computer disposed on the communication network through the network interface 5.

The output section 6 can include a display device such as a liquid crystal display or an organic EL display, or a printer device such as an inkjet printer or a laser printer. For example, the analysis result outputted from the controller 2 may be displayed on a display screen of the display device or printed by the printer.

The input section 7 can be configured to accept an input operation from an operator operating the electrocardiogram analyzing apparatus 1 and output an operation signal correspondingly to the input operation. For example, the input section 7 is a touch panel superimposed and disposed on the display device of the output section 6, an operation button attached to a housing, a mouse, a keyboard, etc.

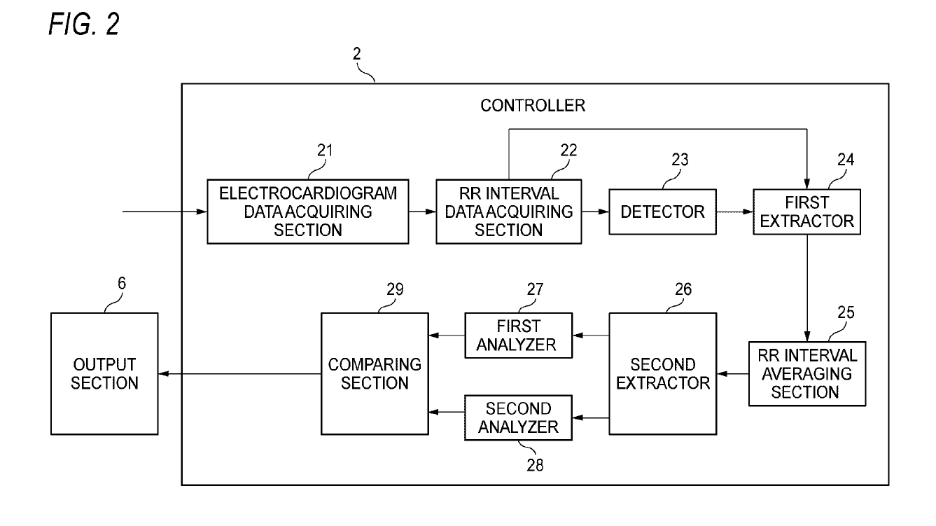

FIG. 2 is a diagram illustrating functional blocks of the controller 2 in the electrocardiogram analyzing apparatus 1 shown in FIG. 1. As shown in FIG. 2, the controller 2 can include an electrocardiogram data acquiring section 21, an RR interval data acquiring section 22, a detector 23, a first extractor 24, an RR interval averaging section 25, a second extractor 26, a first analyzer 27, a second analyzer 28, and a comparing section 29.

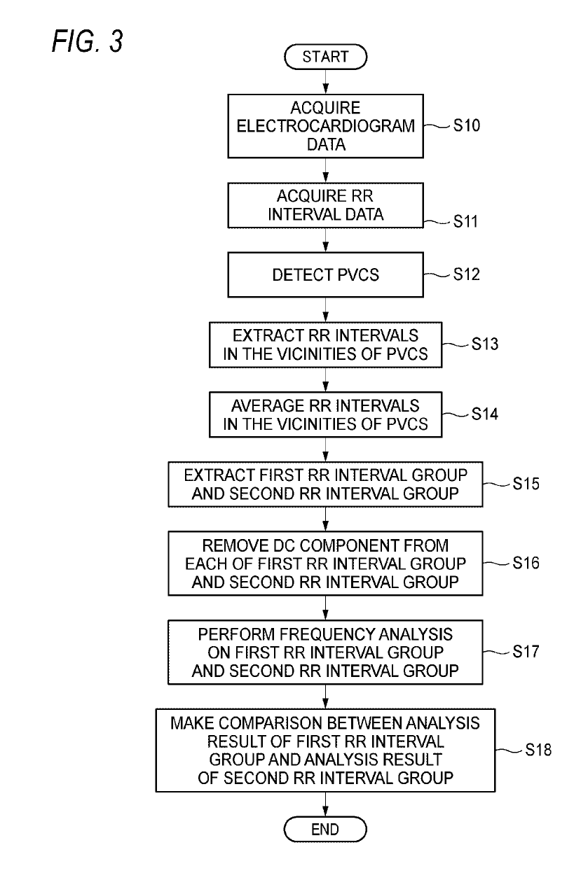

The respective functions of the controller 2 shown in FIG. 2 will be described below with reference to FIG. 3. FIG. 3 is a flow chart for illustrating an electrocardiogram analyzing method executed by the electrocardiogram analyzing apparatus 1 according to the embodiment.

At the beginning, in a step S10 shown in FIG. 3, the electrocardiogram data acquiring section 21 acquires electrocardiogram data stored in the storage 3 (or electrocardiogram data acquired through the sensor interface 4). Next, in a step S11, the RR interval data acquiring section 22 acquires RR interval data including plural RR intervals from the electrocardiogram data acquired by the electrocardiogram data acquiring section 21. Here, RR interval represents an interval between an R wave of one of two adjacent heart beat waveforms and an R wave of the other heart beat waveform, i.e. a time interval between the adjacent heart beat waveforms. For example, the RR interval data may include data pairs in which numbers (hereinafter referred to as beat numbers N) are assigned to all the RR intervals respectively, and each beat number N is paired with a corresponding one of the RR intervals. The beat numbers N may be assigned to the RR intervals in order of appearance of their heart beat waveforms on a time axis.

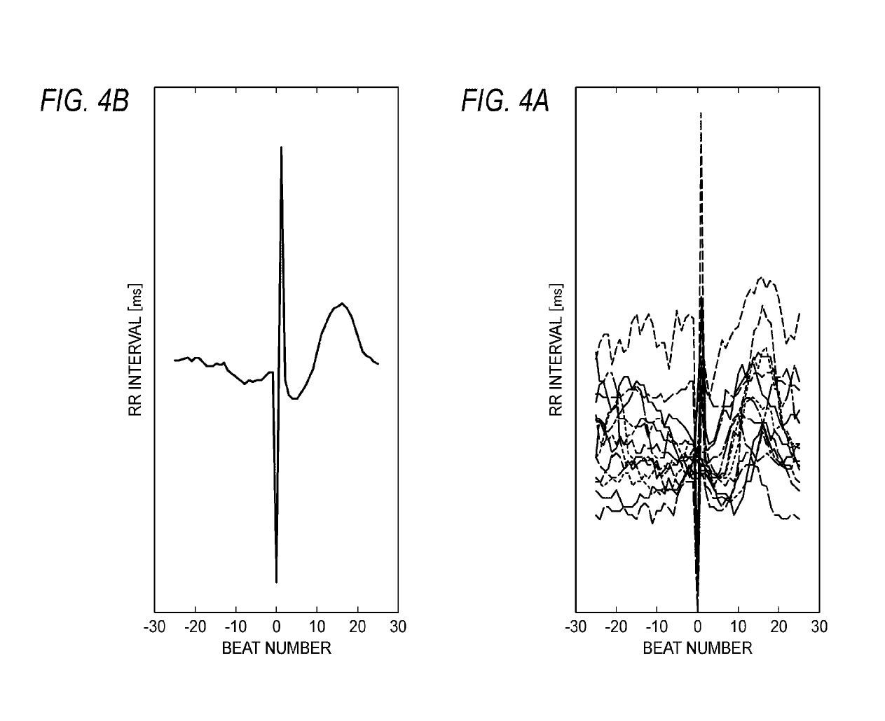

Next, in a step S12, the detector 23 detects plural premature ventricular contractions (hereinafter referred to as PVCs simply) each causing a compensatory pause based on the RR interval data acquired by the RR interval data acquiring section 22. For example, the detector 23 can detect the PVCs based on a variation in the RR intervals corresponding to the beat numbers. When each of the PVCs occurs, the RR intervals vary largely before and after appearance of a V beat (abnormal heart beat waveform) (see FIG. 4A). Therefore, when detecting the large variation in the RR intervals, the detector 23 can detect the PVC. Incidentally, the detector 23 may detect the PVC based on the electrocardiogram data (the shape of an electrocardiogram waveform). In this case, the detector 23 may read a reference waveform of the V beat (hereinafter referred to as PVC reference waveform) stored in advance in the storage 3 and compare the shape of the electrocardiogram waveform with the shape of the PVC reference waveform to thereby detect the PVC. In addition, since there are V beats having various shapes, the V beats having the various shapes may be stored as PVC reference waveforms in the storage 3.

Next, in a step S13, the first extractor 24 extracts, from the RR interval data, RR intervals during occurrence of each of the PVCs detected by the detector 23 and before and after the occurrence of the PVC. For example, as shown in FIG. 4A, an RR interval between a V beat generated due to the PVC and an N beat (normal heart beat waveform) appearing immediately before the V beat is set as an RR interval in a beat number N=0. In addition, an RR interval between the V beat and an N beat appearing immediately after the V beat is set as an RR interval in a beat number N=1. Further, an RR interval between the N beat appearing immediately after the V beat and an N beat appearing immediately after the N beat is set as an RR interval in a beat number N=2. In this manner, the beat numbers N are set with reference to the V beat. In this case, the first extractor 24 totally extracts 51 RR intervals in beat numbers N=−25 to +25. In addition, as shown in FIG. 4A, the first extractor 24 extracts the 51 RR intervals (beat numbers N=−25 to +25) for each of the detected PVCs. Incidentally, in the embodiment, the beat numbers N are set in the range of from −25 to +25 by way of example. The range of the beat numbers N or the number of the RR intervals to be extracted may be changed suitably. In addition, each of the RR intervals extracted for each of the PVCs may be associated with the beat numbers N set with reference to the V beat, as shown in FIGS. 4A and 4B.

Next, in a step S14, the RR interval averaging section 25 obtains an average of the RR intervals extracted for the PVCs for each of the beat numbers N. For example, assume that the RR interval in the beat number N=1 in the PVC appearing in a first time is R¹1, the RR interval in the beat number N=1 in the PVC appearing in a second time is R²1, and the RR interval in the beat number N=1 in the PVC appearing in an m-th (the last) time is R^{m}1. Thus, an average Rav-1 of the RR intervals in the beat number N=1 can be obtained as in the following expression (1). Incidentally, the number of the detected PVCs may be regarded as m here.

Rav-1=(R¹1+R²1 . . . R^{m}1)/m (1)

Similarly, an average Rav-n of the RR intervals in the beat number N=n can be obtained as in the following expression (2).

Rav-n=(R¹n+R²n . . . R^{m}n)/m (2)

In the aforementioned manner, the RR interval averaging section 25 obtains an average of the RR intervals for each of the beat numbers. Accordingly, the averages of the RR intervals associated with the beat numbers respectively can be obtained, as shown in FIG. 4B. Incidentally, the embodiment has been described on the assumption that the PVCs are detected in the step S12. However, when only one PVC is detected, the aforementioned step S14 of obtaining the averages of the plural RR intervals is not performed.

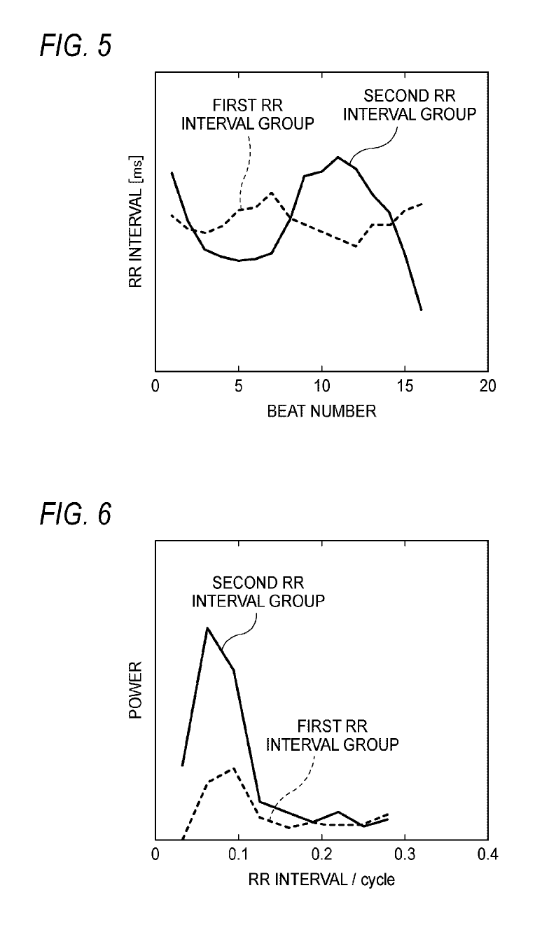

Next, in a step S15, the second extractor 26 extracts a first RR interval group constituted by the plural RR intervals before the occurrence of the PVCs and a second RR interval group constituted by the plural RR intervals after the occurrence of the PVCs respectively from the averages of the RR intervals shown in FIG. 4B. For example, as shown in FIG. 5, the second extractor 26 extracts a first RR interval group (graph line indicated by a dotted line) constituted by the RR intervals in the beat numbers N=−1 to −16 before the occurrence of the PVCs, and a second RR interval group (graph line indicated by a solid line) constituted by the RR intervals in the beat numbers N=+1 to +16 after the occurrence of the PVCs. Incidentally, the range of the beat numbers N to be extracted in the step can be determined desirably. In addition, when only one PVC is detected, the second extractor 26 extracts the first RR interval group constituted by the plural RR intervals before the occurrence of the PVC and the second RR interval group constituted by the plural RR intervals after the occurrence of the PVC respectively. Incidentally, the abscissa of the graph shown in FIG. 5 expresses only the beat numbers of the second RR interval group.

Then, in a step S16, the second extractor 26 removes a DC component from each of the first RR interval group and the second RR interval group. Here, the first RR interval group and the second RR interval group in each of which the DC component has been removed are shown in FIG. 5.

Then, in a step S17, the first analyzer 27 performs frequency analysis using fast Fourier transform (FFT) on the first RR interval group, and the second analyzer 28 performs the frequency analysis using the fast Fourier transform on the second RR interval group. FIG. 6 illustrates a power spectrum (graph line indicated by a dotted line) of the first RR interval group obtained by the first analyzer 27, and a power spectrum (graph line indicated by a solid line) of the second RR interval group obtained by the second analyzer 28. Incidentally, the abscissa of the graph shown in FIG. 6 represents frequency (RR interval/cycle), and the ordinate of the graph represents power.

According to the embodiment, the first RR interval group and the second RR interval group are subjected to be the frequency analysis using the FFT. Thus, results of the frequency analysis can be obtained fast. In addition, although the frequency analysis using the FFT has been described in the embodiment, frequency analysis using another method such as a maximum entropy method (MEM) etc. may be performed.

Finally, in a step S18, the comparing section 29 makes comparison between a first analysis result obtained by the first analyzer 27 and a second analysis result obtained by the second analyzer 28. Specifically, the comparing section 29 calculates a ratio between a total value (integrated value) P1total of powers in respective frequency bands of the power spectrum of the first RR interval group obtained by the first analyzer 27 and a total value (integrated value) P2total of powers in the respective frequency bands of the power spectrum of the second RR interval group obtained by the second analyzer 28. For example, P2total/P1total is about 2.5 in the example shown in FIG. 6.

In addition, the comparing section 29 may calculate a ratio (P2max/P1max) or a difference (P2max−P1max) between a peak value P1max of the powers in the respective frequency bands of the power spectrum of the first RR interval group obtained by the first analyzer 27 and a peak value P2max of the powers in the respective frequency bands of the power spectrum of the second RR interval group obtained by the second analyzer 28.

A comparison result obtained by the comparing section 29 is inputted to the output section 6. For example, the comparison result may be displayed on the display screen of the display device or printed by the printer. Further, each of the graphs shown in FIGS. 4A and 4B and FIGS. 5 and 6 may be displayed on the display device or printed by the printer.

In the example shown in FIG. 6, the ratio (P2total/P1total) of P2total to P1total is about 2.5. In addition, the ratio (P2max/P1max) of P2max to P1max is about 2.9. Further, the difference (P2max−P1max) between P2max and P1max is about 94. All these values are sufficiently large. Based on such a comparison result, a health care professional can judge that an autonomic nerve function such as baroreflex of a patient who has provided the electrocardiogram data is normal.

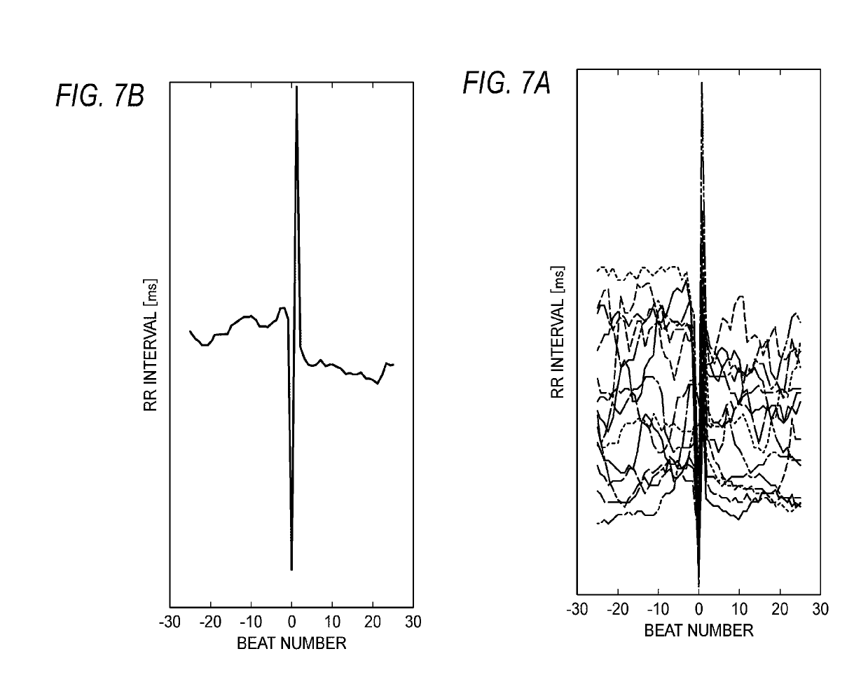

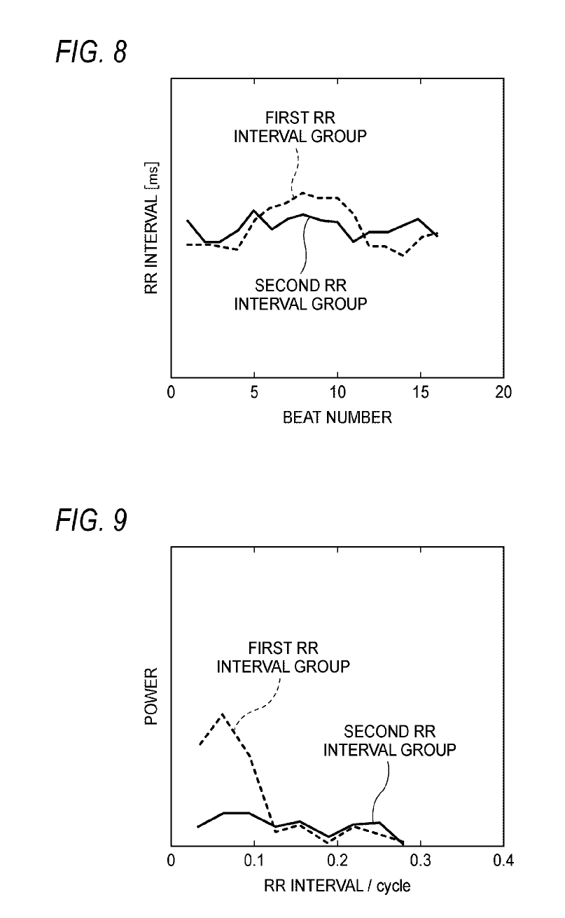

FIGS. 4A and 4B and FIGS. 5 and 6 illustrate the graphs of the patent whose autonomic nerve function is normal. On the other hand, examples of graphs of a patient whose autonomic nerve function is abnormal will be shown as reference examples in FIGS. 7A and 7B and FIGS. 8 and 9. FIG. 7A is a reference graph illustrating the relationship between plural RR intervals in the vicinity of each of PVCs and beat numbers. FIG. 7B is a reference graph illustrating the relationship between the plural RR intervals averaged for each of the beat numbers and the beat numbers. FIG. 8 is a reference graph illustrating graph lines of a first RR interval group and a second RR interval group respectively. FIG. 9 is a reference graph illustrating graph lines of power spectrums of the first RR interval group and the second RR interval group respectively FIGS. 7A and 7B correspond to FIGS. 4A and 4B. FIG. 8 corresponds to FIG. 5. FIG. 9 corresponds to FIG. 6.

In the graph shown in FIG. 9, the power spectrum of the first RR interval group in a low frequency band is larger than the power spectrum of the second RR interval group in the same low frequency band, contrary to the graph shown in FIG. 6. Thus, in the example shown in FIG. 9, a ratio (P2total/P1total) of P2total to P1total is about 0.5 and a ratio (P2max/P1max) of P2max to P1max is small. Further, a difference (P2max−P1max) between P2max and P1max is a negative value. Thus, it can be understood that a significant difference can be observed between the frequency analysis result of the patient whose autonomic nerve function is normal and the frequency analysis result of the patient whose autonomic nerve function is abnormal.

According to the embodiment, plural first RR interval groups constituted by the RR intervals before the occurrence of the PVCs can be subjected to the frequency analysis, and second RR interval groups constituted by the plural RR intervals after the occurrence of the PVCs can be subjected to the frequency analysis. Then, a first analysis result obtained by the first analyzer 27, and a second analysis result obtained by the second analyzer 28 are compared with each other. In this manner, it is possible to provide the electrocardiogram analyzing apparatus 1 or the electrocardiogram analyzing method which can apply frequency analysis to plural RR intervals before occurrence of each PVC and plural RR intervals after the occurrence of the PVC to thereby make it relatively easy to inspect an autonomic nerve function such as baroreflex of a patient.

According to the embodiment, the plural RR intervals extracted for the PVCs are averaged. Then, the first RR interval group and the second RR interval group are extracted respectively from the averaged plural RR intervals, and subjected to the frequency analysis. When the averaging step is provided thus, the frequency analysis does not have to be individually performed on the RR intervals extracted for the PVCs but may be performed only on the averaged RR intervals. Therefore, it is possible to reduce the number of times of calculation of the electrocardiogram analyzing apparatus 1 (or the electrocardiogram analyzing method).

According to the embodiment, the ratio between the total value P1total of the powers in the respective frequency bands of the power spectrum of the first RR interval group and the total value P2total of the powers in the respective frequency bands of the power spectrum of the second RR interval group is calculated. Thus, the first analysis result obtained by the first analyzer 27 and the second analysis result obtained by the second analyzer 28 can be compared with each other.

In addition, the ratio or difference between the peak value P1max of the powers in the respective frequency bands of the power spectrum of the first RR interval group and the peak value P2max of the powers in the respective frequency bands of the power spectrum of the second RR interval group may be calculated. Thus, a first analysis result obtained by the first analyzer 27 and a second analysis result obtained by the second analyzer 28 can be compared with each other.

In this manner, it is possible to provide the electrocardiogram analyzing apparatus 1 which can inspect an autonomic nerve function such as baroreflex of a patient relatively easily. Further, according to the electrocardiogram analyzing apparatus 1 according to the embodiment, HRT is evaluated directly. Thus, it is possible to evaluate the autonomic nerve function such as baroreflex with high accuracy.

In order to carry out the electrocardiogram analyzing apparatus 1 according to the embodiment by software, the electrocardiogram analyzing program may be incorporated in the storage 3 or the ROM in advance. In addition, the electrocardiogram analyzing program may be stored in a computer-readable medium such as a magnetic disk (an HDD or a floppy (trademark) disk), an optical disk (a CD-ROM, a DVD-ROM, a Blu-ray (trademark) disk, or the like), a magneto-optical disk (an MO, or the like), a flash memory (an SD card, a USB memory, an SSD, or the like), or the like. In this case, when the computer-readable medium is connected to the electrocardiogram analyzing apparatus 1, the electrocardiogram analyzing program stored in the storage medium can be incorporated into the storage 3. When the program incorporated in the storage 3 is uploaded onto the RAM and the processor executes the uploaded program, the controller 2 can execute the various processes shown in FIG. 2. In other words, when the program is executed by the processor, the controller 2 functions as the electrocardiogram data acquiring section 21, the RR interval data acquiring section 22, the detector 23, the first extractor 24, the RR interval averaging section 25, the second extractor 26, the first analyzer 27, the second analyzer 28 and the comparing section 29, respectively.

The electrocardiogram analyzing program may be downloaded from a computer on a communication network through the network interface 5. Also in this case, the downloaded program can be similarly incorporated into the storage 3.

Although the embodiment of the present disclosure has been described above, the technical scope of the invention should not be limitedly interpreted based on the description of the embodiment. The embodiment is merely an example. It should be understood by those skilled in the art that change can be made on the embodiment variously within the scope of the claimed invention. The technical scope of the invention should be defined based on the scope of the claimed invention and the scope of any equivalent thereto.

This application is based on Japanese Patent Application No. 2015-178764 filed on Sep. 10, 2015, the entire contents of which are incorporated herein by reference.

What is claimed is:

1. An electrocardiogram analyzing method comprising:
(a) acquiring electrocardiogram data representing an electrocardiogram waveform having heart beat waveforms occurring continuously on a time axis;
(b) acquiring RR interval data from the electrocardiogram data, wherein the RR interval data includes RR intervals, and each of the RR interval represents a time interval between adjacent ones of the heart beat waveforms;
(c) detecting at least one premature ventricular contraction causing a compensatory pause;
(d) extracting, from the RR interval data, RR intervals during occurrence of the detected premature ventricular contraction and before and after the occurrence of the detected premature ventricular contraction;
(e) extracting a first RR interval group and a second interval group respectively from the RR intervals, wherein the first RR interval group includes the RR intervals before the occurrence of the premature ventricular contraction, and the second RR interval group includes the RR intervals after the occurrence of the premature ventricular contraction;
(f) performing a predetermined frequency analysis on the first RR interval group;
(g) performing the predetermined frequency analysis on the second RR interval group; and
(h) making a comparison between a first analysis result obtained by the step (f) and a second analysis result obtained by the step (g).

2. The method of claim 1,
wherein
the step (c) comprises detecting premature ventricular contractions each causing the compensatory pause, and
the step (d) comprises extracting, from the RR interval data, RR intervals during occurrence of each of the detected premature ventricular contractions and before and after the occurrence of each of the detected premature ventricular contracts,
wherein the electrocardiogram analyzing method further comprises:
(i) averaging the RR intervals extracted for each of the premature ventricular contractions, and
wherein the step (e) comprises extracting, from the averaged RR intervals, the first RR interval group and the second interval group respectively.

3. The method of claim 1, wherein the predetermined frequency analysis is a frequency analysis using fast Fourier transform (FFT).

4. The method according to claim 2, wherein
in the step (i), the RR intervals extracted for each of the premature ventricular contractions are associated respectively with orders of heart beat waveforms, which are set with reference to an abnormal heart beat waveform generated due to the premature ventricular contraction, and
the RR intervals are averaged for each of the orders of the heart beat waveforms.

5. The method according to of claim 1, wherein the step (h) comprises calculating a ratio between a total value of powers in respective frequency bands obtained by the step (f) and a total value of powers in the respective frequency bands obtained by the step (g).

6. The method according to claim 1, wherein the step (h) comprises calculating a ratio or difference between a peak value of powers in respective frequency bands obtained by the step (f) and a peak value of powers in the respective frequency bands obtained by the step (g).

7. An electrocardiogram analyzing apparatus comprising:
an electrocardiogram data acquiring section configured to acquire electrocardiogram data representing an electrocardiogram waveform having heart beat waveforms occurring continuously on a time axis;
an RR interval data acquiring section configured to acquire RR interval data from the electrocardiogram data, wherein the RR interval data includes RR intervals, and each of the RR interval represents a time interval between adjacent ones of the heart beat waveforms;
a detector configured to detect at least one premature ventricular contraction causing a compensatory pause;
a first extractor configured to extract, from the RR interval data, RR intervals during occurrence of the detected premature ventricular contraction and before and after the occurrence of the detected premature ventricular contraction;
a second extractor configured to extract a first RR interval group and a second interval group respectively from the RR intervals, wherein the first RR interval group includes the RR intervals before the occurrence of the premature ventricular contraction, and the second RR interval group includes the RR intervals after the occurrence of the premature ventricular contraction;
a first analyzer configured to perform a predetermined frequency analysis on the first RR interval group;
a second analyzer configured to perform the predetermined frequency analysis on the second RR interval group; and
a comparing section configured to make a comparison between a first analysis result obtained by the first analyzer and a second analysis result obtained by the second analyzer.

8. The apparatus of claim 7,
wherein
the detector is configured to detect premature ventricular contractions each causing the compensatory pause, and
the first extractor is configured to extract, from the RR interval data, RR intervals during occurrence of each of the detected premature ventricular contractions and before and after the occurrence of each of the detected premature ventricular contracts,
wherein the electrocardiogram analyzing apparatus further comprises:
an RR interval averaging section configured to average the RR intervals extracted for each of the premature ventricular contractions, and
wherein the second extractor is configured to extract, from the averaged RR intervals, the first RR interval group and the second interval group respectively.

9. The apparatus of claim 7, wherein the predetermined frequency analysis is a frequency analysis using fast Fourier transform (FFT).

10. The apparatus of claim 8, wherein
the RR intervals extracted for each of the premature ventricular contractions are associated respectively with orders of heart beat waveforms, which are set with reference to an abnormal heart beat waveform generated due to the premature ventricular contraction, and
the RR interval averaging section is configured to average the RR intervals for each of the orders of the heart beat waveforms.

11. The apparatus of claim 7, wherein the comparing section is configured to calculate a ratio between a total value of powers in respective frequency bands obtained by the first analyzer and a total value of powers in the respective frequency bands obtained by the second analyzer.

12. The apparatus of claim 7, wherein the comparing section is configured to calculate a ratio or difference between a peak value of powers in respective frequency bands obtained by the first analyzer and a peak value of powers in the respective frequency bands obtained by the second analyzer.

13. A computer readable medium storing an electrocardiogram analyzing program causing a computer to perform operations comprising:
(a) acquiring electrocardiogram data representing an electrocardiogram waveform having heart beat waveforms occurring continuously on a time axis;
(b) acquiring RR interval data from the electrocardiogram data, wherein the RR interval data includes RR intervals, and each of the RR interval represents a time interval between adjacent ones of the heart beat waveforms;
(c) detecting at least one premature ventricular contraction causing a compensatory pause;
(d) extracting, from the RR interval data, RR intervals during occurrence of the detected premature ventricular contraction and before and after the occurrence of the detected premature ventricular contraction;
(e) extracting a first RR interval group and a second interval group respectively from the RR intervals, wherein the first RR interval group includes the RR intervals before the occurrence of the premature ventricular contraction, and the second RR interval group includes the RR intervals after the occurrence of the premature ventricular contraction;
(f) performing a predetermined frequency analysis on the first RR interval group;
(g) performing the predetermined frequency analysis on the second RR interval group; and
(h) making a comparison between a first analysis result obtained by the step (f) and a second analysis result obtained by the step (g).

14. (canceled)
